(19) United States
(12) Patent Application Publication
(10) Pub. No.: US 2008/0220106 A1
(43) Pub. Date: Sep. 11, 2008

(54) Otological Solutions Containing Lapacho Extracts

(75) Inventor: Mario Miscioscia, Torino (IT)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Euro-Pharma S.r.l., Torino (IT)

(21) Appl. No.: 11/996,844

(22) PCT Filed: Jul. 28, 2006
(86) PCT No.: PCT/EP06/64780

§ 371 Date: Jan. 25, 2008

(30) Foreign Application Priority Data
Jul. 29, 2005 (IT) TO2005A000533

(51) Int. Cl. A61K 36/00; A61P 27/16

(52) U.S. Cl. 424/775; 424/725

(57) ABSTRACT

The present invention refers to otological solutions containing Lapacho extracts, and their preparation and uses.

### FIELD OF THE INVENTION

The present invention refers to products for the body hygiene, and in particular to otological solutions, comprising basically Lapacho extracts in polar or non-polar solvents.

The invention refers also to the preparation of such products and their use for body hygiene.

### STATE OF THE ART

Tabebuia impetiginosa (a.k.a T. avellanedae) is tree species in the family Bignoniaceae (catalpa family) native to rainforests throughout Central and South America.

Mainly exported from Argentina, Paraguay, and Brazil, it is commonly known as: lapacho, pau d'arco, taheebo, and ipe roxo.

Since more than one thousand years the plant is used for medical purposes by indigenous populations which utilize in particular the internal part of the bark obtaining extracts useful in the treatment of various diseases.

Many studies have been performed in order to identify the pharmacological potential of the Lapacho extracts and various active principle have been isolated. Essentially 18 different quinones have been identified (see Edward H. Oswald—British Journal of Phytotherapy, Vol. 3, n. 3 (1993/1994).

The pharmacological properties of various extracts have been studied and it was proved that they posses antibiotic, antiviral, antimicotic, and antiseptic activity which made them appropriated i.e. in the treatment of mouth, nose and throat infections.

### DETAILED DESCRIPTION OF THE INVENTION

It was now surprisingly found that the traditional extracts of Lapacho bark, either obtained with polar or non polar solvents, yield the particular property to disaggregate, solubilise and remove earwax and, therefore, they can be used in otological solutions useful for the cleansing of the auricular duct.

According to the present invention, these products can be easily prepared through hot-extraction with polar solvents (such as water, alcohol, acetone) or non polar solvents (such as ether, etc.) or their mixtures, of the Lapacho bark and then by filtering the suspension (following well-known methods).

When preferred, quinones contained in the Lapacho bark (which are not water soluble, i.e., quercetin) could be added to the water extract obtained from the bark, according to the method described in the Italian patent no. 1.304.304 of the same applicant.

According to this latter patent, water-insoluble quinones can be added to the aqueous solution either as commercially available products or recovering them from the bark after the extraction with hot-water, for example by salifying them in order to make them soluble in water as widely described in the above said patent.

The latter instance obviously presents the advantage that all naturally present quinones contained in the Lapacho bark are available, even those not commercially available.

The obtained product is then processed following the known techniques to obtain the final mixture ready to be used.

Usually, the extract is concentrated up to about 10%; however, different concentrations could be used.

Besides Lapacho extract, the otological formulations described in the present invention can also contain other active ingredients, such as anti-inflammatory and/or antiseptic ingredients together with possible preservatives (i.e., nipagine).

Cosmetic excipients known in the otological field (in particular those used for the cleansing of the auricular duct) can be added to the above solution, such as glycerine, urea peroxide, PEG, sodium lauret sarcosinate, citric acid, dibasic sodium phosphate, polysorbytan monostearate, hydroxypropylmethyl cellulose, etc.

When preferred, the solution containing Lapacho extract can be added with other vegetal extracts, such as camomile, almond oil, other herbal extracts with anti-inflammatory activity, Flavonoids, vitamins, etc.

Otological formulations prepared according to the present invention can comprise, for example:

| | |
|---|---|
| Lapacho extract | 5%-90%, |
| water | 5%-90%, |
| herbal extracts | 5%-90%. |

A formulation according to the present invention may comprise, for example:

| | |
|---|---|
| Lapacho extract | 10% |
| water | 89.8% |
| Nipagine | 0.2% |

Sodium hydroxyde q.b. a pH 6.5

The present invention will then better described with the following examples.

### EXAMPLE 1

10 kg micronised Lapacho bark (Tabebuia species) are warmed at 65-70° C. for 1-3 hours in 90 kg aqueous solvent (controlled pH value=7.5) under continuous air-free stirring. Thereafter the solution is cooled at 20° C., then filtered. Filtrate is collected and preservatives (0.2% paraben mixture) are added. The above solution can be utilised to prepare final formulations.

### EXAMPLE 2

10 kg micronised Lapacho bark (Tabebuia species) are warmed at 65-70° C. for 1-3 hours in 90 kg aqueous solvent (controlled pH value =7.5) under continuous air-free stirring. Thereafter the solution is cooled at 20° C., then filtered. Filtrate is collected and preservatives (0.2% paraben mixture) are added. The above solution is then added with ethanolic and/or glycerolic and/or lyophilic extracts at a concentration of 1-70% (p/p). The above solution can be utilised to prepare final formulations.

As reported previously, Lapacho extract can be prepared by means of solvents different from water (either polar or non polar) or by means of appropriate solvent mixtures (either polar or non polar), in analogy to what has been described above and using methods already described in the scientific literature.

The solution obtained according the above procedures can be formulated in the form of drops, bottles and/or strips containing variable amounts of liquid, different sprays with or without gaseous propellant, to compliance any preferred method of use.

1. Otological formulations containing Lapacho extract.

2. Products according to claim 1 where said extracts are obtained by treating Lapacho bark with polar solvents.

3. Products according to claim 1 where said extracts are obtained by treating Lapacho bark with non polar solvents.

4. Products according to claim 2 where said extract is added with water-insoluble quinones.

5. Products according to claim 1 which also comprise anti-inflammatory, antiseptic products, vegetable extracts, flavonoids, vitamins, preservatives.

6. Products according to claim 1 including:

| | |
|---|---|
| Lapacho extract | 5%-90%, |
| water | 5%-90%, |
| vegetable extracts | 5%-90%. |

7. Products according to claim 1 including:

| | |
|---|---|
| Lapacho extract | 10.0% |
| water | 89.8% |
| nipagine | 0.2% |

NaOH up to pH 6.5

8. (canceled)

9. Products according to claim 1 in the form of drops, bottles and/or strips containing variable amounts of liquid, different sprays with or without gaseous propellant, rubber-syringes either disposable or not.

10. A method for dissolving, disaggregating and removing ear wax in the auricular duct by means of the products described in claim 1.
